(19) United States
(12) Patent Application Publication
(10) Pub. No.: US 2024/0058587 A1
(43) Pub. Date: Feb. 22, 2024

(54) MEDICAL DEVICES AND RELATED METHODS

(71) Applicant: Boston Scientific Scimed, Inc., Maple Grove, MN (US)

(72) Inventors: Adam Joseph ROYER, Brooklyn Park, MN (US); Jeffrey S. LINDQUIST, Maple Grove, MN (US); Eve GEORGE, Waltham, MA (US); Daryl Lee MARTIN, Plymouth, MN (US); Alan BEELER, Jr., Albertville, MN (US); Lane JACOBS, Fruit Heights, UT (US)

(73) Assignee: Boston Scientific Scimed, Inc., Maple Grove, MN (US)

(21) Appl. No.: 18/450,075

(22) Filed: Aug. 15, 2023

Related U.S. Application Data
(60) Provisional application No. 63/398,587, filed on Aug. 17, 2022.

(51) Int. Cl. A61M 25/10

(52) U.S. Cl. CPC A61M 25/10; A61M 2025/1075; A61M 2025/1084

(57) ABSTRACT

Balloon catheters are described, including a balloon catheter that includes an inflatable balloon and a braiding positioned on an exterior surface of the inflatable balloon. The inflatable balloon includes an intermediate portion, a distal waist, and a transition portion between the intermediate portion and the distal waist. At least a portion of the transition portion has a cone angle of at least 40 degrees. The braiding includes a plurality of threads, and a spacing between adjacent threads of the plurality of threads over the distal waist is smaller than a spacing between adjacent threads of the plurality of threads over the intermediate portion. A spacing between adjacent threads of the plurality of threads over the transition portion gradually transitions over the transition portion from the spacing between adjacent threads over the intermediate portion to the spacing between adjacent threads over the distal waist.

### CROSS-REFERENCE TO RELATED APPLICATIONS

This application claims priority to U.S. Provisional Application No. 63/398,587, filed on Aug. 17, 2022, which is incorporated by reference herein in its entirety.

### TECHNICAL FIELD

Various aspects of the present disclosure relate generally to medical devices and related methods. More specifically, the present disclosure relates to medical devices and related methods for positioning and/or expanding a balloon for urological procedures, for example, such as percutaneous nephrolithotomy or nephrolithotripsy (PCNL).

### BACKGROUND

Many medical procedures that once required open surgery can now be done with less invasive techniques that limit the size of incision, thus reducing recovery time and risk of infection. In the field of urology, for example, renal calculi or kidney stones can accumulate in the urinary tract and become lodged in the kidney. Kidney stones are deposits of materials from urine, typically minerals and acid salts. While smaller stones may pass from the body naturally, larger stones may require surgical intervention for removal. Although open surgery was once the standard treatment for the removal of stones, other less invasive techniques, such as ureteroscopy and percutaneous nephrolithotomy or nephrolithotripsy (hereinafter “PCNL”), have emerged as safer, effective alternatives. PCNL procedures often involve delivering a sheath (i.e., a renal sheath) to the kidney to help remove one or more kidney stones. The sheath may be delivered to the one or more kidney stones via a balloon catheter. However, the size, shape, thickness, etc. of the balloon catheter may limit the proximity of the sheath to the one or more kidney stones.

The devices and methods of the current disclosure may rectify some of the deficiencies described above or address other aspects of the prior art.

### SUMMARY

Examples of the present disclosure relate to, among other things, medical devices and methods. Each of the examples disclosed herein may include one or more of the features described in connection with any of the other disclosed examples.

In one example, a balloon catheter may include an inflatable balloon and a braiding positioned on an exterior surface of the inflatable balloon. The inflatable balloon may include an intermediate portion, a distal waist, and a transition portion between the intermediate portion and the distal waist. At least a portion of the transition portion may have a cone angle of at least 40 degrees. The braiding may include a plurality of threads, and a spacing between adjacent threads of the plurality of threads over the distal waist may be smaller than a spacing between adjacent threads of the plurality of threads over the intermediate portion. A spacing between adjacent threads of the plurality of threads over the transition portion may gradually transition over the transition portion from the spacing between adjacent threads over the intermediate portion to the spacing between adjacent threads over the distal waist.

The balloon catheter may include one or more of the following aspects. The plurality of threads may include a plurality of clockwise threads, a plurality of counterclockwise threads, and a plurality of longitudinal threads. The plurality of clockwise threads, the plurality of counterclockwise threads, and the plurality of longitudinal threads may be braided together over the exterior surface of the inflatable balloon. The plurality of threads may include the same number of clockwise threads, counterclockwise threads, and longitudinal threads over the intermediate portion, the transition portion, and the distal waist. The balloon catheter may further include a thermoplastic polyurethane layer formed over at least a portion of the inflatable balloon between the inflatable balloon and the braiding. The thermoplastic polyurethane layer may be a first thermoplastic polyurethane layer, and the balloon catheter may further include a second thermoplastic polyurethane layer formed over at least a portion of the braiding. The balloon catheter may further include a silicone-based lubricious coating on an exterior of the balloon catheter.

The inflatable balloon may include an inner balloon wall and an outer balloon wall. The outer balloon wall may be configured to be spaced away from the inner balloon wall over at least the intermediate portion and the transition portion when the inflatable balloon is inflated. The balloon catheter may further include a mandrel, and the inner balloon wall may be coupled to the mandrel. The inflatable balloon may include a diameter of 10 mm, and the transition portion may include a length of 8.5 mm. At least a portion of the transition portion may extend at an angle of 25 degrees from the distal end of the intermediate portion. The transition portion may be a distal transition portion, and the inflatable balloon may further include a proximal waist and a proximal transition portion between the intermediate portion and the proximal waist.

The balloon catheter may be configured to be used for a percutaneous nephrolithotomy or nephrolithotripsy procedure such that the distal waist is positioned adjacent to a stone. The plurality of threads may be fused together over at least a portion of the distal waist. The plurality of threads may be or comprise Vectran™ fibers. The balloon catheter may have a rated burst pressure of at least 30 atmospheres.

In another aspect, a method of forming a balloon catheter may include forming a base balloon, inflating and/or sealing the base balloon, and delivering and forming a braiding on an exterior surface of the base balloon. The base balloon may include an inner balloon wall and an outer balloon wall spaced apart from the inner balloon wall when the base balloon is inflated. The base balloon may include an intermediate portion, a distal waist, and a transition portion between the intermediate portion and the distal waist. At least a portion of the transition portion may include a cone angle of at least 40 degrees when inflated. The braiding may include a plurality of clockwise threads, a plurality of counterclockwise threads, and a plurality of longitudinal threads. A spacing between adjacent threads of the plurality of threads over the distal waist may be smaller than a spacing between adjacent threads of the plurality of threads over the intermediate portion. A spacing between adjacent threads of the plurality of threads over the transition portion may gradually transition over the transition portion from the spacing between adjacent threads over the intermediate portion to the spacing between adjacent threads over the distal waist.

The method may include one or more of the following aspects. The braiding may be formed with a braider machine and by moving the base balloon at different speeds corresponding to a braiding angle and/or the portion of the base balloon that is receiving the braiding. The plurality of threads may include the same number of clockwise threads, counterclockwise threads, and longitudinal threads over the intermediate portion, the transition portion, and the distal waist. The method may further include, before delivering and forming the braiding on the exterior of the base balloon, treating the exterior surface of the balloon, and, after delivering and forming the braiding on the exterior of the base balloon, treating an exterior surface of the braided balloon.

In yet another aspect, a balloon catheter may include an inflatable balloon and a braiding positioned on an exterior surface of the inflatable balloon. The inflatable balloon may include an intermediate portion, a proximal waist, a proximal transition portion between the proximal waist and the intermediate portion, a distal waist, and a distal transition portion between the intermediate portion and the distal waist. At least a portion of the distal transition portion may include a cone angle of at least 50 degrees. The braiding may include a plurality of clockwise threads, a plurality of counterclockwise threads, and a plurality of longitudinal threads. A spacing between adjacent threads of the plurality of threads over the distal waist may be smaller than a spacing between adjacent threads of the plurality of threads over the intermediate portion. A spacing between adjacent threads of the plurality of threads over the transition portion may gradually transition over the transition portion from the spacing between adjacent threads over the intermediate portion to the spacing between adjacent threads over the distal waist.

The balloon catheter may include one or more of the following aspects. The plurality of threads may include the same number of clockwise threads, counterclockwise threads, and longitudinal threads over the proximal waist, the proximal transition portion, the intermediate portion, the distal transition portion, and the distal waist.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this application, illustrate exemplary features of the present disclosure and together with the description, serve to explain the principles of the disclosure.

FIG. 1 illustrates an exemplary medical device.

FIG. 2 illustrates a distal portion of the medical device of FIG. 1, without braiding.

FIG. 3A illustrates the distal portion of the medical device of FIG. 1, with braiding, and FIG. 3B is a lateral cross-section view of a portion of the medical device of FIG. 1 with the braiding.

FIG. 4 is a flow diagram of an exemplary method of forming the medical device of FIG. 1, according to aspects of this disclosure.

### DETAILED DESCRIPTION

Examples of this disclosure include devices and methods for facilitating and/or improving the efficacy, efficiency, cost, and/or safety of a medical procedure. Embodiments of the disclosure may relate to devices and methods for performing various medical procedures and/or treating portions of the kidneys, ureters, bladder, urethra, or any other portion of the urinary tract. Additionally, embodiments of the disclosure may relate to devices and methods for performing various medical procedures and/or treating portions of the large intestine (colon), small intestine, cecum, esophagus, stomach, or any other portion of the gastrointestinal tract or biliary tree. Furthermore, embodiments of the disclosure may relate to devices and methods for performing various medical procedures and/or treating portions of anatomy, for example, accessed via a body lumen, such as the larynx, trachea, bronchi (primary bronchi), lobar (secondary bronchi), segmental (tertiary bronchi), or any other portion of the respiratory system. In these aspects, the devices and methods discussed herein may be used to treat any other suitable subject anatomy (collectively referred to herein as a “treatment site”).

Various embodiments described herein include single-use or disposable medical devices. Some aspects of the disclosure may be used in performing an endoscopic, arthroscopic, bronchoscopic, ureteroscopic, colonoscopic, or other type of procedure. For example, the disclosed aspects may be used with ureteroscopes, endoscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, catheters, diagnostic or therapeutic tools or devices, or any other suitable delivery device or medical device, for example, for treatment through a body lumen. Alternatively, various embodiments described herein may be delivered to a treatment site alone and/or used separate from another scope or medical device. One or more of the elements discussed herein could be metallic, plastic, or include a shape memory metal (such as Nitinol), a shape memory polymer, a polymer, or any combination of biocompatible materials.

Reference will now be made in detail to examples of the present disclosure described above and illustrated in the accompanying drawings. Wherever possible, the same reference numbers will be used throughout the drawings to refer to the same or like parts. It is noted that one or more aspects of the medical systems, devices, and methods discussed herein may be combined and/or used with one or more aspects of other medical systems, devices, and methods discussed herein.

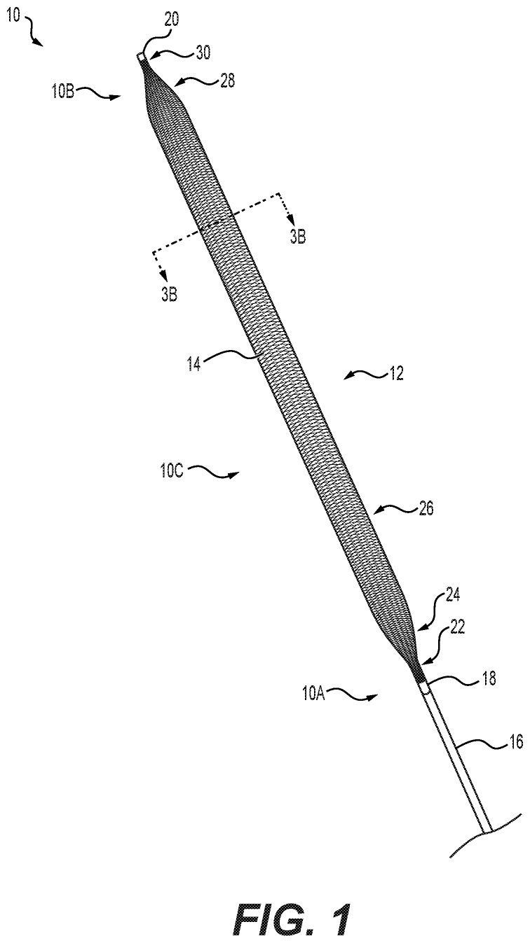

FIG. 1 illustrates a medical device, for example, an exemplary balloon catheter 10. Balloon catheter 10 includes an expandable balloon 12 (balloon 12) and a fiber braiding 14 (braiding 14), for example, on an outer surface or exterior of balloon 12. Balloon catheter 10 may include a proximal portion 10A, a distal portion 10B, and an intermediate portion 10C, for example, between proximal portion 10A and distal portion 10B. As discussed in detail below, balloon catheter 10 may be used to access one or more treatment sites, for example, to be positioned near or adjacent (i.e., proximal) to one or more kidney stones. Moreover, the shape, orientation, and/or arrangement of braiding 14 (i.e., the fibers that form fiber braiding 14) may vary over various portions of balloon 12, for example, to help support and retain balloon 12 when balloon 12 is inflated.

Balloon catheter 10 may also include or be coupled to a mandrel 16, for example, that extends from proximal portion 10A of balloon catheter 10 to distal portion 10B of balloon catheter 10. Mandrel 16 may include a proximal block 18, for example, abutting a proximal-most end of a proximal waist 22 of balloon 12, and mandrel 16 may also include a distal block 20, for example, abutting a distalmost end of distal portion 10B of balloon catheter 10. In some aspects, proximal block 18 and/or distal block 20 may be formed by radially extending portions of mandrel 16. Furthermore, balloon 12 may include proximal waist 22, for example, coupling proximal waist 22 of balloon 12 to mandrel 16. Balloon 12 includes a proximal transition portion or proximal transition zone 24, for example, expanding from proximal waist 22 to an intermediate or barrel portion 26 of balloon 12. Furthermore, balloon 12 includes a distal transition portion or distal transition zone 28, for example, tapering from intermediate portion 26 to a distal waist 30 of balloon 12. Distal waist 30 may couple distal portion 10B of balloon catheter 10 to mandrel 16. Proximal transition zone 24 and distal transition zone 28 may each be substantially conical.

As shown in FIG. 1, braiding 14 may have different fiber densities over various portions of balloon 12, for example, over various portions of proximal portion 10A, distal portion 10B, and intermediate portion 10C of balloon catheter 10. For example, braiding 14 may have a first density when wound around proximal waist 22. Braiding 14 may have a second density when wound around intermediate portion 26, with the second density being less dense than the first density. Braiding 14 may gradually transition from the first density to the second density, for example, over at least a portion of proximal transition zone 24. Additionally, braiding 14 may have a third density when wound around distal waist 30. The third density may be denser than the second density, for example, the same or a substantially similar density as the first density of braiding 14 over proximal waist 22. Braiding 14 may gradually transition from the second density to the third density, for example, over at least a portion of distal transition zone 28.

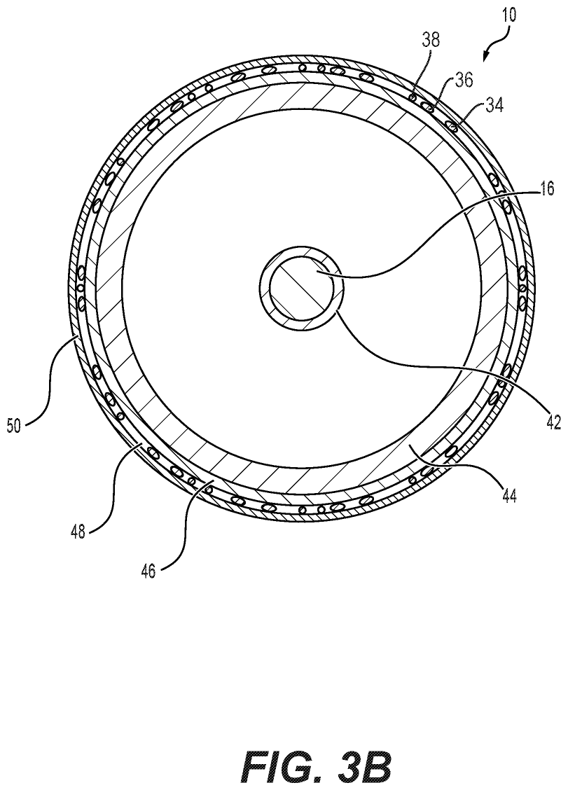

As discussed below with respect to, for example, FIGS. 3A and 3B, braiding 14 may include a plurality of fibers extending in a clockwise spiral, for example, when viewed from a distal end of balloon catheter 10, and a plurality of fibers extending in a counterclockwise spiral, for example, when viewed from the distal end of balloon catheter 10. Additionally, braiding 14 may include a plurality of fibers extending longitudinally along respective portions of balloon 12. The clockwise spiral fibers, the counterclockwise spiral fibers, and the longitudinal fibers may be interwoven (see FIG. 3A) to form braiding 14. Furthermore, the spacing between respective fibers (i.e., between adjacent clockwise fibers, between adjacent counterclockwise fibers, and between respective longitudinal fibers) may vary between different portions of balloon catheter 10, for example, to form the respective braiding densities discussed above.

As discussed herein, balloon catheter 10 may be used for percutaneous nephrolithotomy or nephrolithotripsy (PCNL) to help break-up, remove, or otherwise treat one or more kidney stones. In these aspects, balloon catheter 10 may be used to deliver a renal sheath, for example, through an incision in the patient, and to the patient's kidney. Balloon catheter 10 may be used to deliver the renal sheath to a position proximate to one or more kidney stones. In other aspect, balloon catheter 10 may be used in one or more other procedures, for example, endoscopic retrograde cholangiopancreatography (ERCP), balloon and laser angioplasty, nephrostomy, electrode placement, etc. In these aspects, balloon catheter 10 may be used, for example, to help remove or otherwise treat ureteral stones, bladder stones, gallstones, bile duct stones, polyps, stent placement, gastroenteral anastomosis, choledochoduodenostomy, etc., and/or to help deliver one or more other sheaths to a position within a patient.

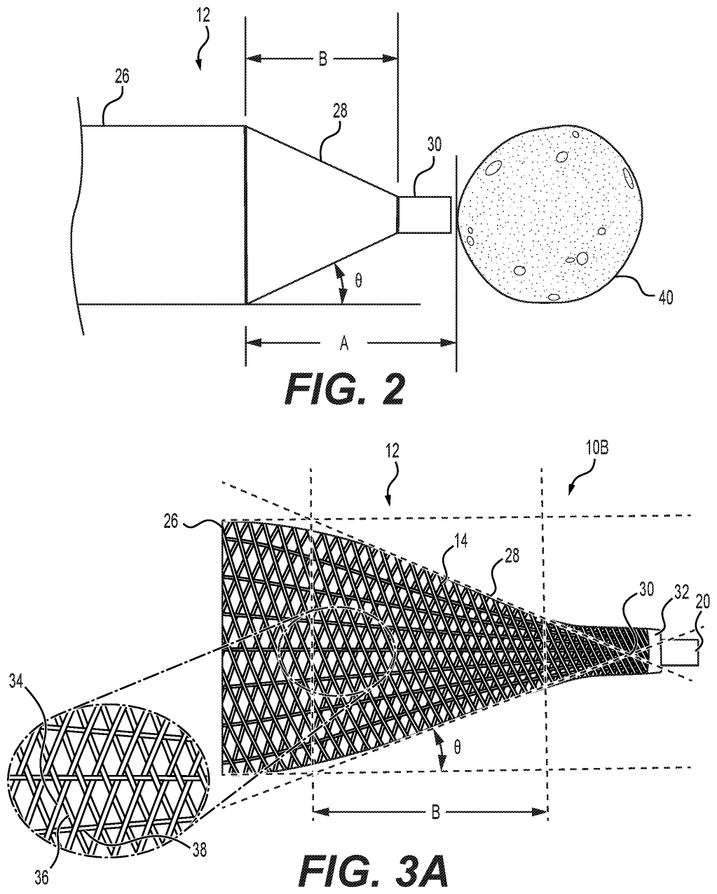

FIG. 2 illustrates a distal portion of balloon 12. FIG. 2 omits braiding 14 for clarity. As shown, the distal portion of balloon 12 includes a portion of intermediate portion 26, distal transition zone 28, and distal waist 30. Additionally, as shown, distal waist 30 may be positioned proximate to a target site and/or an object 40. In this aspect, object 40 is a stone (e.g., a kidney stone). Nevertheless, as mentioned above, object 40 may be another type of object or tissue to be accessed, removed, and/or otherwise treated with the help of balloon catheter 10.

As shown in FIG. 2, when distal waist 30 is positioned proximate to object 40, intermediate portion 26 is spaced apart from object 40 by a distance A. For example, balloon 12 may include a cone angle of approximately 50 degrees over at least a portion of distal transition zone 28. In this aspect, distal transition zone 28 may taper in diameter between intermediate portion 26 and distal waist 30. Moreover, distal transition zone 28 may taper inward, for example, at an angle Θ of approximately 20 degrees or greater, for example, approximately 25 degrees, relative to an imaginary line extending longitudinally and/or tangentially from intermediate portion 26. Accordingly, distal transition zone 28 may form a cone angle (i.e., two times angle Θ) of approximately 40 degrees or greater, for example, approximately 50 degrees. In these aspects, balloon 10 may be a 10 mm diameter balloon, and distal transition zone 28 may include a longitudinal length B, for example, approximately 7-10 mm, for example, approximately 8.5 mm. Additionally, when balloon catheter 10 is assembled with balloon 12 inflated and distal waist 30 positioned adjacent to object 40, a distalmost end of intermediate portion 26 may be positioned approximately 12-15 mm, for example, approximately 13.5 mm, from object 40 (i.e., distance A). In these aspects, distal waist 30, distal block 20, and/or a distal portion of mandrel 16 may account for the additional spacing between the distalmost end of intermediate portion 26 and object 40.

Although distal waist 30 is shown in FIG. 2 as being spaced apart from object 40 by a small distance, in some aspects, distal waist 30 (and/or distal block 20 or a distal portion of mandrel 16, not shown in FIG. 2) may be in abutting contact with object 40. Additionally, although the junction or transition between intermediate portion 26 and distal transition zone 28, and the junction or transition between distal transition zone 28 and distal waist 30, are each shown as discrete junctions or transitions, this disclosure is not so limited. For example, as shown in FIG. 3A, balloon 12, and thus balloon catheter 10, may include gradual junctions or transitions between intermediate portion 26 and distal transition zone 28, and also between distal transition zone 28 and distal waist 30.

FIG. 3A illustrates distal portion 10B of balloon catheter 10, including balloon 12 and braiding 14. As shown in FIG. 3A, junctions or transitions between intermediate portion 26 and distal transition zone 28, and also between distal transition zone 28 and distal waist 30, may be gradual. Additionally, distal transition zone 28 may extend at an angle, for example, angle θ (e.g., greater than approximately 20 degrees, for example, approximately 25 degrees), from intermediate portion 26, and thus forming a cone angle greater than approximately 40 degrees, for example, approximately 50 degrees. As mentioned above, distal transition zone 28 may include longitudinal length B, for example, approximately 7-10 mm, for example, approximately 8.5 mm. Furthermore, braiding 14 may distally terminate at a distal coupling 32, for example, at a distal portion of distal waist 30. For example, braiding 14 may be fused together and/or to distal waist 30 at distal coupling 32.

As mentioned above, braiding 14 may include a plurality of fibers extending in a clockwise spiral (clockwise fibers 34), for example, when viewed from a distal end of balloon catheter 10, and a plurality of fibers extending in a counterclockwise spiral (counterclockwise fibers 36), for example, when viewed from the distal end of balloon catheter 10. In some aspects, braiding 14 may include approximately sixteen clockwise fibers 34 and approximately sixteen counterclockwise fibers 36. Additionally, braiding 14 may include a plurality of fibers extending longitudinally (longitudinal fibers 38) along respective portions of balloon 12. In some aspects, braiding 14 may include approximately sixteen longitudinal fibers 38.

Clockwise fibers 34, counterclockwise fibers 36, and longitudinal fibers 38 may be interwoven (see FIG. 3A) to form braiding 14. For example, braiding 14 may be formed by a braider machine, for example, with a variable braiding angle, as discussed below. In some aspects, one or more of clockwise fibers 34, the counterclockwise fibers 36, and longitudinal fibers 38 may be or comprise Vectran™ fibers. Furthermore, one or more portions of braiding 14 may be formed with the braider machine, and by moving balloon 12 at various different speeds, for example, depending on the braiding angle and/or the portion of balloon 12 that is receiving braiding 14. For example, in some aspects, the braider machine may deliver braiding 14 at similar or the same braiding angle over various different portions of balloon 12. However, balloon 12 may be advanced or otherwise moved relative to the braider machine at various different speeds, thereby imparting various different spacings between adjacent threads 34, 36, and 38 in braiding 14 over different portions of balloon 12. In these aspects, braiding 14 (and thus balloon catheter 10) includes the same number of clockwise threads 34, counterclockwise threads 36, and longitudinal threads 38 over the entirety of balloon catheter 10, for example, over intermediate portion 26, distal transition zone 28, and distal waist 30 of balloon 12.

Furthermore, the spacing between respective fibers (i.e., between adjacent clockwise fibers 34, between adjacent counterclockwise fibers 36, and between respective longitudinal fibers 38) may vary between different portions of balloon catheter 10, for example, to form the respective braiding densities discussed above. For example, as shown in FIGS. 1 and 3A, clockwise fibers 34, counterclockwise fibers 36, and longitudinal fibers 38 may be spaced apart and interwoven at a first spacing and/or a first interval over intermediate portion 26 of balloon 12. For example, clockwise fibers 34 and counterclockwise fibers 36 may be braided at a braid angle between approximately 55 degrees and approximately 75 degrees, for example, between approximately 60 degrees and approximately 70 degrees, for example, approximately 65 degrees, over intermediate portion 26 of balloon 12. Additionally, clockwise fibers 34, counterclockwise fibers 36, and longitudinal fibers 38 may be spaced apart and interwoven at a second spacing and/or a second interval over distal waist 30 of balloon 12. For example, clockwise fibers 34 and counterclockwise fibers 36 may be braided at a braid angle between approximately 35 degrees and approximately 55 degrees, for example, between approximately 40 to approximately 50 degrees, for example, approximately 45 degrees, over distal waist 30 of balloon 12.

The spacing and interval at which clockwise fibers 34, counterclockwise fibers 36, and longitudinal fibers 38 are interwoven may gradually transition over distal transition zone 28, for example, from the spacing and/or interval over intermediate portion 26 of balloon 10 to the spacing and/or interval over distal waist 30 of balloon 12. It is noted that longitudinal fibers 38 may extend along the outer surface of balloon 12 parallel to the longitudinal axis of balloon 12. However, the spacing between adjacent longitudinal fibers 38 over distal waist 30 is smaller than the spacing between adjacent longitudinal fibers 38 over intermediate portion 26, for example, gradually transitioning between the respective spacings over distal transition zone 28.

FIG. 3B is a cross-sectional view of a portion of balloon catheter 10, for example, over a portion of intermediate portion 26 (FIG. 1). As shown, an inner balloon wall 42 may surround a portion of mandrel 16. Additionally, an outer balloon wall 44 may be radially spaced away from inner balloon wall 42, for example, when balloon 12 is inflated. Inner balloon wall 42 and/or outer balloon wall 44 may be formed of an elastomeric material, for example, including a flexible polyether and a rigid polyamide (e.g., Pebax®). A base layer 46 may be deposited or otherwise positioned radially outside of outer balloon wall 44. Base layer 46 may be formed of a thermoplastic polyurethane, and may help fibers 34, 36, and 38 adhere to balloon 12 and/or maintain relative positions on balloon 12.

Additionally, a fiber layer 48 may be deposited or otherwise positioned radially outside of base layer 46. For example, as shown, fiber layer 48 may include a plurality of clockwise fibers 34, a plurality of counterclockwise fibers 36, and a plurality of longitudinal fibers 38, as discussed above, to form braiding 14.

Moreover, an outer layer 50 may be deposited or otherwise positioned radially outside of fiber layer 48. Outer layer 50 may be formed of thermoplastic polyurethane, and may help to enclose or embed fibers 34, 36, and 38. Although outer layer 50 is shown radially outside of fiber layer 48 (i.e., embedded fibers 34, 36, and 38), it is noted that outer layer 50 may also propagate between fibers 34, 36, 38, for example, filling the space between adjacent fibers 34, 36, 38 and/or abutting one or more portions of base layer 46. Additionally, in some aspects, outer layer 50 may be at least partially lubricious, for example, a silicone-based lubricious coating, which may help delivery of balloon catheter 10, for example, by helping to prevent adhesion to balloon catheter 10. Alternatively or additionally, an additional outer coating (not shown) may be applied to at least a portion of outer layer 50. The outer coating may also be at least partially lubricious, for example, a silicone-based lubricious coating, which may help delivery of balloon catheter 10, for example, by helping to prevent adhesion (i.e., adhesion of tissue) to balloon catheter 10. In some aspects, the outer coating may only be applied to one or more portions of intermediate portion 10C of balloon catheter 10 (i.e., the barrel portion), which may help delivery of balloon catheter 10, for example, by helping to prevent adhesion to intermediate portion 10C while still maintaining at least some adhesive properties of distal portion 10B of balloon catheter 10.

Furthermore, in some aspects, balloon catheter 10, for example, over one or more portions of intermediate portion 10C, may have a cross-sectional diameter of between approximately 4 mm and approximately 14 mm, for example, approximately 10 mm. Additionally, in some aspects, balloon catheter 10, for example, over one or more portions of distal portion 10B, may have a cross-sectional diameter of between approximately 1.5 mm to approximately 3 mm, for example, between approximately 2.1 mm and approximately 2.8 mm, for example, approximately 2.35 mm.

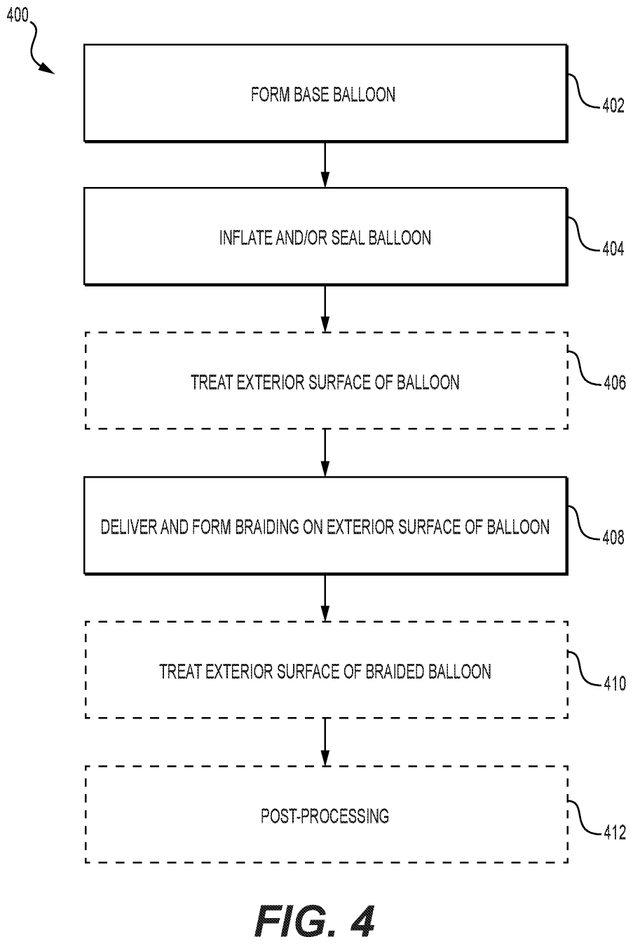

FIG. 4 illustrates an exemplary method 400 that may be performed to form balloon catheter 10. In these aspects, a first step 402 includes forming a base balloon. As discussed above, balloon 12 may include inner balloon wall 42 and outer balloon wall 44. One or more of inner balloon wall 42 and outer balloon wall 44 may be formed by one or more extrusions. One or more of inner balloon wall 42 and outer balloon wall 44 may be stretched, molded, and/or trimmed, for example, in order to form the desired size and/or shape of balloon 12. Inner balloon wall 42 may be coupled to, or otherwise positioned around, mandrel 16. Outer balloon wall 44 may be coupled to inner balloon wall 42, for example, at proximal and distal ends, allowing for outer balloon wall 44 to expand. In some aspects, as shown in FIG. 3B, outer balloon wall 44 may include a larger radial thickness than inner balloon wall 42, for example, to help maintain the shape and/or structure of balloon 12 when inflated. As mentioned above, inner balloon wall 42 and/or outer balloon wall 44 may be formed of an elastomeric material, for example, including a flexible polyether and a rigid polyamide (e.g., Pebax®).

Next, a step 404 includes inflating and/or sealing balloon 12. For example, air may be delivered to the area between inner balloon wall 42 and outer balloon wall 44 to help inflate balloon 12. Additionally or alternatively, proximal and/or distal portions of inner balloon wall 42 and outer balloon wall 44 may be coupled together and/or otherwise sealed.

In an optional step 406, method 400 may include treating the exterior surface of balloon 12. For example, optional step 406 may include one or more of a plasma treatment, a base coating (e.g., a thermoplastic polyurethane coating), and/or annealing the base coating. In this aspect, optional step 406 may include depositing base layer 46 on an outside of outer balloon wall 44, and base layer 46 may help fibers 34, 36, and 38 adhere to balloon 12 and/or maintain relative positions on balloon 12.

Next, method 400 includes a step 408 that includes delivering and forming braiding 14 on the outer surface or exterior of balloon 12. As discussed above, braiding 14 (i.e., including clockwise fibers 34, the counterclockwise fibers 36, and longitudinal fibers 38) may be deposited on the exterior surface of outer balloon wall 44, for example by a braider machine (e.g., with a variable braiding angle) to form fiber layer 48. In some aspects, one or more of clockwise fibers 34, the counterclockwise fibers 36, and longitudinal fibers 38 may be or comprise Vectran™ fibers, and the spacing between adjacent fibers 34, 36, 38 may vary over different portions of balloon 12. Furthermore, one or more portions of braiding 14 may be formed with the braider machine, and by moving balloon 12 at various different speeds, for example, depending on the braiding angle and/or the portion of balloon 12 that is receiving braiding 14. For example, in some aspects, the braider machine may deliver braiding 14 at similar or the same braiding angle over various different portions of balloon 12. However, balloon 12 may be advanced or otherwise moved relative to the braider machine at various different speeds, thereby imparting various different spacings between adjacent threads 34, 36, and 38 in braiding 14 over different portions of balloon 12. In these aspects, balloon 12 may be manually advanced relative to the braider machine, or may be automatically and/or robotically advanced relative to the braider machine.

In some aspects, method 400 may include an optional step 410, which includes treating the exterior surface of the braided balloon (i.e., balloon 12 with braiding 14). In these aspects, optional step 410 may include one or more of a plasma treatment, a top coating (e.g., a thermoplastic polyurethane coating), and/or annealing the top coating. In this aspect, optional step 410 may include depositing outer layer 50 on the outside of fiber layer 48. Outer layer 50 may be formed of thermoplastic polyurethane, and may help to enclose or embed fibers 34, 36, and 38. As mentioned above, in some aspects, outer layer 50 may be at least partially lubricious, for example, a silicone-based lubricious coating, which may help prevent adhesion to balloon catheter 10. Alternatively or additionally, an additional outer coating (not shown) may be applied to at least a portion of outer layer 50. The outer coating may also be at least partially lubricious, for example, a silicone-based lubricious coating, which may help prevent adhesion to balloon catheter 10. In some aspects, the outer coating may only be applied to one or more portions of intermediate portion 10C of balloon catheter 10 (i.e., the barrel portion), which may help prevent adhesion to intermediate portion 10C while still maintaining at least some adhesion properties of distal portion 10B of balloon catheter 10.

Lastly, in some aspects, method 400 may include an optional step 412, which includes one or more post-processing steps or procedures. In these aspects, optional step 412 may include one or more of extruding an inner shaft and/or inner support (i.e., mandrel 16), bonding or otherwise coupling balloon 12 to the inner shaft (e.g., coupling or positioning inner balloon wall 42 onto mandrel 16), trimming balloon catheter 10, assembling or otherwise coupling balloon catheter 10 to one or more other components (i.e., for delivering and/or inflating balloon catheter 10), and/or bonding one or more portions of balloon catheter 10 (e.g., laser bonding and/or UV manifold bonding one or more portions of balloon catheter 10, for example, proximal and/or distal portions of balloon catheter 10). Alternatively or additionally, in some aspects, optional step 412 may include one or more of checking the pressure of balloon catheter 10 (e.g., by inflating balloon catheter 10 to one or more desired pressures to ensure structural consistency and/or stability), applying one or more coatings to one or more portions of the exterior of balloon catheter 10 (e.g., Glidex™ hydrophilic or Mediglide™ silicone coatings on distal portion of balloon catheter 10), folding balloon catheter 10, positioning balloon catheter 10 within a balloon protector, positioning balloon catheter 10 within a packaging, and/or sterilizing balloon catheter 10 (e.g., by exposure to sterilizing heat (e.g., steam or dry heat), radiation (e.g., UV radiation), ethylene oxide gas, vaporized hydrogen peroxide, etc.

Various aspects of the medical devices and related methods discussed herein may help to provide an ultra-high pressure and/or non-compliant balloon catheter 10, for example, for performing a PCNL procedure. Additionally, various aspects of the medical devices and related methods may help to provide for an exterior fiber braiding, with variable spacing, which may help to provide for a high burst strength, while also providing for a small distal cone and/or a small overall profile. For example, braiding 14 on balloon 12 may help to allow for a rated burst pressure of approximately 30 atmospheres or higher for balloon catheter 10. Furthermore, distal transition zone 28 may form a short distal conical section, having a cone angle of greater than 40 degrees, for example, greater than 45 degrees (e.g., approximately 50 degrees). As shown in FIGS. 2 and 3A, the cone angle may be two times angle Θ. For example, Θ may be greater than 20 degrees, for example, greater than 22.5 degrees (e.g., approximately 25 degrees), such that the cone angle is greater than 40 degrees, for example, greater than 45 degrees (e.g., approximately 50 degrees). Distal transition zone 28 may allow for balloon catheter 10 to be positioned adjacent to or otherwise in close proximity to one or more objects 40 (i.e., a kidney stone), allowing for easier and/or safer manipulation, energy application, removal, or other treatments. As discussed above, braiding 14 may be denser (i.e., adjacent fibers 34, 36, 38 may be closer together over distal transition zone 28 than intermediate portion 10C), which may further increase the rated burst pressure of balloon catheter 10, as many balloon catheters burst over one or more distal and/or tapered sections when inflated to higher pressures.

While principles of the present disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

1. A balloon catheter, comprising:
an inflatable balloon, wherein the inflatable balloon includes an intermediate portion, a distal waist, and a transition portion between the intermediate portion and the distal waist, wherein at least a portion of the transition portion has a cone angle of at least 40 degrees; and
a braiding positioned on an exterior surface of the inflatable balloon, wherein the braiding includes a plurality of threads, wherein a spacing between adjacent threads of the plurality of threads over the distal waist is smaller than a spacing between adjacent threads of the plurality of threads over the intermediate portion, and wherein a spacing between adjacent threads of the plurality of threads over the transition portion gradually transitions over the transition portion from the spacing between adjacent threads over the intermediate portion to the spacing between adjacent threads over the distal waist.

2. The balloon catheter of claim 1, wherein the plurality of threads includes a plurality of clockwise threads, a plurality of counterclockwise threads, and a plurality of longitudinal threads, wherein the plurality of clockwise threads, the plurality of counterclockwise threads, and the plurality of longitudinal threads are braided together over the exterior surface of the inflatable balloon.

3. The balloon catheter of claim 2, wherein the plurality of threads includes the same number of clockwise threads, counterclockwise threads, and longitudinal threads over the intermediate portion, the transition portion, and the distal waist.

4. The balloon catheter of claim 3, further comprising a thermoplastic polyurethane layer formed over at least a portion of the inflatable balloon between the inflatable balloon and the braiding.

5. The balloon catheter of claim 4, wherein the thermoplastic polyurethane layer is a first thermoplastic polyurethane layer, and wherein the balloon catheter further comprises a second thermoplastic polyurethane layer formed over at least a portion of the braiding.

6. The balloon catheter of claim 5, further comprising a silicone-based lubricious coating on an exterior of the balloon catheter.

7. The balloon catheter of claim 1, wherein the inflatable balloon includes an inner balloon wall and an outer balloon wall, and wherein the outer balloon wall is configured to be spaced away from the inner balloon wall over at least the intermediate portion and the transition portion when the inflatable balloon is inflated.

8. The balloon catheter of claim 7, further comprising a mandrel, wherein the inner balloon wall is coupled to the mandrel.

9. The balloon catheter of claim 1, wherein the inflatable balloon includes a diameter of 10 mm, and wherein the transition portion includes a length of 8.5 mm.

10. The balloon catheter of claim 9, wherein at least a portion of the transition portion extends at an angle of 25 degrees from the distal end of the intermediate portion.

11. The balloon catheter of claim 10, wherein the transition portion is a distal transition portion, and wherein the inflatable balloon further includes a proximal waist and a proximal transition portion between the intermediate portion and the proximal waist.

12. The balloon catheter of claim 1, wherein the balloon catheter is configured to be used for a percutaneous nephrolithotomy or nephrolithotripsy procedure such that the distal waist is positioned adjacent to a stone.

13. The balloon catheter of claim 1, wherein the plurality of threads are fused together over at least a portion of the distal waist.

14. The balloon catheter of claim 1, wherein the plurality of threads are Vectran™ fibers.

15. The balloon catheter of claim 1, wherein the balloon catheter has a rated burst pressure of at least 30 atmospheres.

16. A method of forming a balloon catheter, comprising:
forming a base balloon, wherein the base balloon includes an inner balloon wall and an outer balloon wall spaced apart from the inner balloon wall when the base balloon is inflated;
inflating and/or sealing the base balloon, wherein base balloon includes an intermediate portion, a distal waist, and a transition portion between the intermediate portion and the distal waist, wherein at least a portion of the transition portion includes a cone angle of at least 40 degrees when inflated; and
delivering and forming a braiding on an exterior surface of the base balloon, wherein the braiding includes a plurality of clockwise threads, a plurality of counterclockwise threads, and a plurality of longitudinal threads, wherein a spacing between adjacent threads of the plurality of threads over the distal waist is smaller than a spacing between adjacent threads of the plurality of threads over the intermediate portion, and wherein a spacing between adjacent threads of the plurality of threads over the transition portion gradually transitions over the transition portion from the spacing between adjacent threads over the intermediate portion to the spacing between adjacent threads over the distal waist.

17. The method of claim 16, wherein the braiding is formed with a braider machine and by moving the base balloon at different speeds corresponding to a braiding angle and/or the portion of the base balloon that is receiving the braiding, and
wherein the plurality of threads includes the same number of clockwise threads, counterclockwise threads, and longitudinal threads over the intermediate portion, the transition portion, and the distal waist.

18. The method of claim 17, further comprising:
before delivering and forming the braiding on the exterior of the base balloon, treating the exterior surface of the balloon; and
after delivering and forming the braiding on the exterior of the base balloon, treating an exterior surface of the braided balloon.

19. A balloon catheter, comprising:
an inflatable balloon, wherein the inflatable balloon includes an intermediate portion, a proximal waist, a proximal transition portion between the proximal waist and the intermediate portion, a distal waist, and a distal transition portion between the intermediate portion and the distal waist, wherein at least a portion of the distal transition portion includes a cone angle of at least 50 degrees; and
a braiding positioned on an exterior surface of the inflatable balloon, wherein the braiding includes a plurality of clockwise threads, a plurality of counterclockwise threads, and a plurality of longitudinal threads, wherein a spacing between adjacent threads of the plurality of threads over the distal waist is smaller than a spacing between adjacent threads of the plurality of threads over the intermediate portion, and wherein a spacing between adjacent threads of the plurality of threads over the transition portion gradually transitions over the transition portion from the spacing between adjacent threads over the intermediate portion to the spacing between adjacent threads over the distal waist.

20. The balloon catheter of claim 19, wherein the plurality of threads includes the same number of clockwise threads, counterclockwise threads, and longitudinal threads over the proximal waist, the proximal transition portion, the intermediate portion, the distal transition portion, and the distal waist.
